(19) United States
(12) Patent Application Publication
(10) Pub. No.: US 2008/0214944 A1
(43) Pub. Date: Sep. 4, 2008

(54) SYSTEM, APPARATUS AND METHOD FOR MOBILE REAL-TIME FEEDBACK BASED ON CHANGES IN THE HEART TO ENHANCE COGNITIVE BEHAVIORAL THERAPY FOR ANGER OR STRESS REDUCTION

(76) Inventors: Margaret E. Morris, Portland, OR (US); Farzin Guilak, Beaverton, OR (US); Michael E. Labhard, Lake Oswego, OR (US); Mick J. Flanigan, Beaverton, OR (US); Richard Sloan, New York, NY (US); Larry Jamner, Laguna Beach, CA (US); Ethan Gorenstein, Metuchen, NJ (US)

Correspondence Address: INTEL CORPORATION;c/o INTELLEVATE, LLC, P.O. BOX 52050, MINNEAPOLIS, MN, 55402, US

(21) Appl. No.: 12/028,691

(22) Filed: Feb. 8, 2008

Related U.S. Application Data
(60) Provisional application No. 60/900,483, filed on Feb. 9, 2007.
(60) Provisional application No. 60/900,484, filed on Feb. 9, 2007.

(51) Int. Cl. A61B 5/0402

(52) U.S. Cl. 600/509

(57) ABSTRACT

A system, apparatus and method for mobile real-time feedback based on changes in the heart to enhance cognitive behavioral therapy for anger or stress reduction. In an embodiment, an apparatus receives data relating to a heart of an individual from at least one input device. The apparatus provides a mobile intervention via at least one output device and measures the effectiveness of the mobile intervention in reducing anger or stress in the individual. Other embodiments are described and claimed.

### CROSS-REFERENCE TO RELATED APPLICATIONS

This application is related to pending U.S. patent application Ser. No. 11/641,973, filed on Dec. 20, 2006, and entitled “Apparatus for Monitoring Physiological, Activity and Environmental Data,” by inventors Margaret Morris, Terry Dishongh and Farzin Guilak. This application is related to pending U.S. patent application Ser. No. 11/704,703, filed on Feb. 9, 2007, and entitled “System, Apparatus and Method for Emotional Experience Time Sampling via a Mobile Graphical User Interface,” by inventor Margaret Morris. This application is related to and claims priority to pending U.S. Provisional Patent Application No. 60/900,483, filed on Feb. 9, 2007, and entitled “System, Apparatus and Method for Real-Time Health Feedback on a Mobile Device Based on Physiological, Contextual and Self-Monitored Indicators of Mental and Physical Health States,” by inventors Margaret Morris et al. This application is related to and claims priority to pending U.S. Provisional Patent Application No. 60/900,484, filed on Feb. 9, 2007, and entitled “System, Apparatus and Method for Mobile Real-Time Feedback Based on Changes In Heart Rate Variability to Enhance Cognitive Behavioral Therapy for Anger or Stress Reduction,” by inventors Mick J. Flanigan et. al. This application is related to pending U.S. patent application Ser. No. ______, filed on ______, and entitled “System, Apparatus and Method for Real-Time Health Feedback on a Mobile Device Based on Physiological, Contextual and Self-Monitored Indicators of Mental and Physical Health States,” by inventors Margaret Morris et al.

### BACKGROUND

Stress, particularly interpersonal hostility, is a known risk factor for cardiovascular disease. Hostility is marked by decreased heart rate variability, a physiological marker that can be detected by ECG. Psychotherapy protocols have been designed to help individuals prone to conflict. These protocols are based on cognitive behavioral therapy. Cognitive behavioral therapy (“CBT”) has three chief components: cognitive reframing, behavioral changes and physiological relaxation.

### BRIEF DESCRIPTION OF THE DRAWINGS

FIG. 1 illustrates one embodiment of a system for mobile real-time feedback based on changes in the heart to enhance cognitive behavioral therapy for anger or stress reduction.

FIG. 2 illustrates one embodiment of an apparatus for mobile real-time feedback based on heart data.

FIG. 3 illustrates one embodiment of a mobile therapies module.

FIG. 4 illustrates one embodiment of a logic flow for mobile real-time feedback based on changes in the heart to enhance cognitive behavioral therapy for anger or stress reduction.

FIG. 5 illustrates the dynamic relationship between continuous monitoring and feedback in the embedded assessment approach.

### DETAILED DESCRIPTION

Emotional health is intimately intertwined with physical health. Cardiovascular disease progression is influenced by an array of stressors, including hostility or proneness to interpersonal conflict. Psychological and behavioral interventions can help people modulate interpersonal stress and its consequences. A cognitive behavioral therapy is a psychotherapy based on critically evaluating and modifying everyday thoughts and behaviors, with the aim of positively influencing emotional and physical health.

Embedded assessment is a technology design strategy to drive preventive health care and early disease detection. In embedded assessment techniques, devices for collecting biometric data and assessing an individual's emotional and/or physiological state are integrated into an individual's surroundings and the devices regularly used by the individual. Health monitoring may be then translated into personalized feedback. The feedback may support immediate wellness and long-term disease prevention. By embedding one or more assessment techniques into an individual's surroundings and/or activities, it becomes easier to collect relevant data about the individual's physiological and mental state over significant periods of time. Further, by making assessments through embedded sensors or devices, the individual avoids stigmas and negative perceptions associated with poor health.

Embodiments of the present invention provide a real-time mobile coaching system that is responsive to changes in the heart and enhances cognitive behavioral therapy for anger or stress reduction. The mobile coaching system may help individuals whose emotional reactivity poses a risk for certain diseases, such as coronary artery disease, depression, strokes, headaches, pain disorders, psychiatric concerns, and so forth. Thus, mobile therapies or interventions are triggered by moment-to-moment changes in an individual's physical state and emotional health and are made available when and where they are most needed. A mobile intervention is administered via a mobile device that provides therapeutic feedback to the individual on a real-time basis either right before, during or directly after feelings of anger or stress.

Embodiments of the present invention may help to improve emotional regulation and limit the cumulative toll of feelings of anger or stress in an individual. Therapeutic feedback and interventions may support immediate wellness and long-term disease prevention.

Various embodiments of the present invention may be generally directed to a system, apparatus and method for mobile real-time feedback based on changes in the heart to enhance cognitive behavioral therapy for anger or stress reduction. Other embodiments may be described and claimed.

Various embodiments may comprise one or more elements or components. An element may comprise any structure arranged to perform certain operations. Each element may be implemented as hardware, software, or any combination thereof, as desired for a given set of design parameters or performance constraints. Although an embodiment may be described with a limited number of elements in a certain topology by way of example, the embodiment may include more or less elements in alternate topologies as desired for a given implementation. It is worthy to note that any reference to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment.

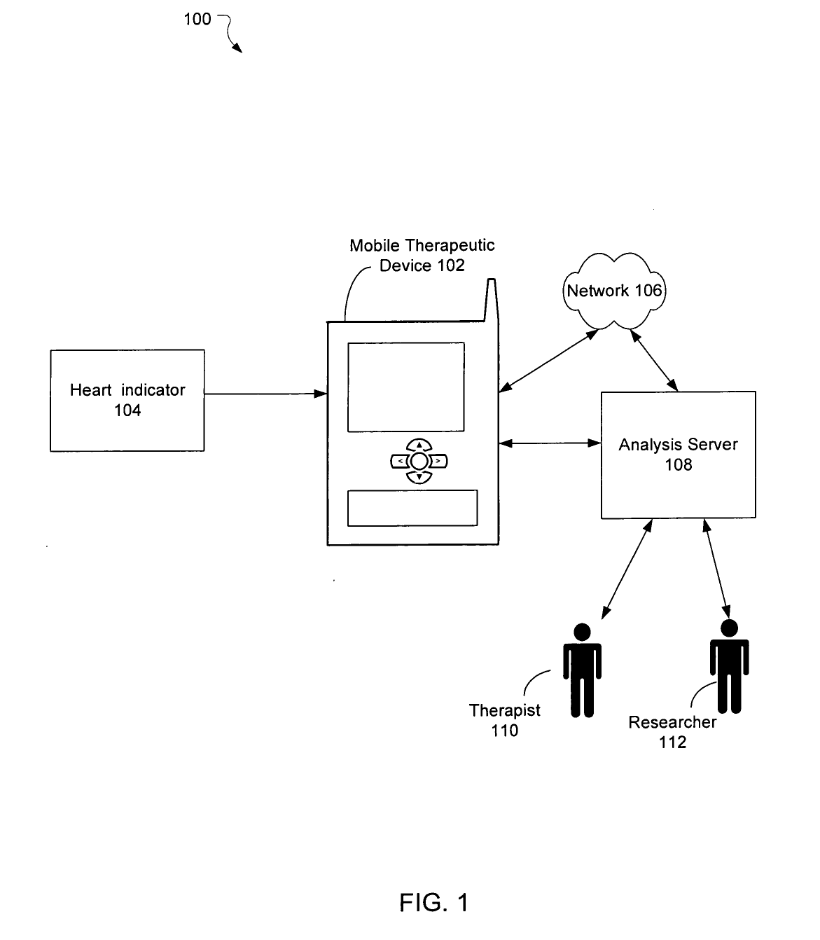

FIG. 1 illustrates one embodiment of a system 100 for mobile real-time feedback based on changes in the heart (e.g., heart rate variability, ECG amplitude, ST segment analysis, QT interval, etc.) to enhance cognitive behavioral therapy for anger or stress reduction. In one embodiment, system 100 comprises a mobile therapeutic device 102, a heart indicator 104, a network 106, an analysis server 108, a therapist 110 and a researcher 112.

At a high level and in an embodiment, real-time data is continuously collected for an individual via a body-worn monitoring system capable of capturing heart data (e.g., heart rate variability, ECG amplitude, ST segment analysis, QT interval, etc.). An example of such a device is a wireless ECG monitor worn on the chest to measure a broad array of cardiovascular characteristics (e.g., heart indicator 104). The collected data are transmitted to mobile therapeutic device 102. Device 102 processes the data to determine physiological stress that may reflect anger in the individual. The data may also be processed by heart indicator 104 (e.g., ECG device) and/or by analysis server 108. If physiological stress is detected, mobile therapeutic device 102 determines and delivers a sequence of interactive events to provide appropriate therapy or intervention to enhance cognitive behavioral therapy for anger or stress reduction. The administered mobile intervention via device 102 provides therapeutic feedback to the individual on a real-time basis either right before, during or directly after detection of the physiological stress indicators in the individual.

There are different stages of stress. The process flow for the mobile interventions is organized so someone can indicate by self-initiation (or through physiological monitoring) where they are in the different stages of stress. In embodiments, the invention provides for different sequences of interactions or interventions based on the different stages of stress. For example, for one stage of stress an intervention such as an exit strategy or breathing exercise may be administered. For another stage of stress, an intervention such as cognitive reframing and problem solving may be administered.

In embodiments of the invention, journaling or self-report of health states and related behaviors occur through a variety of modalities including responses to questionnaires, touching of iconic images, gesturing, menu selection, and touch screen activation. These modalities data or feedback to the mobile health device 102 may be done via direct or indirect input into device 102. These health journaling techniques include, but are not necessarily limited to, menu selection of adjectives to indicate emotional and physical health states, social context and behaviors (e.g. eating, exercise, sleeping), menu selection of dietary intake, camera documentation of context, the “panic button” means of initiating the mobile intervention, selection of images to represent health state or situation, etc. Items can be selected via touch screen, navigation buttons, a scroll dial or a stylus, for example.

This health journaling or self-report data can be gathered via a method of experience time sampling (ETS) in which the device prompts users for responses at frequent time intervals (for example every 30 minutes). Experience time sampling allows researchers to assess health states and behavior frequently (and very close in time/proximal to) events in daily life, rather than asking people to report retrospectively. Many studies have demonstrated extreme inaccuracy in retrospective self-report. The intuitive, less intrusive and more expedient translation of questionnaires for mobile health device 102 described herein is more suitable for frequent administration and therefore facilitates frequent health surveying (e.g., ETS). Experience time sampling can also be triggered by physiological health indicators (such as changes in the heart), contextual factors (stressful appointments or meetings, entered manually or automatically detected via synchronization with an online calendar), location (via beacons, GPS, etc.), time of day (morning and evening journaling), or upon completion of mobile therapy interventions (asking mood after mobile interventions).

Further, data may be collected regarding the effectiveness of the administered intervention and the individual's pattern of reacting to and recovering from feelings of stress or anger. Based on the determined effectiveness of the administered mobile intervention, mobile interventions may be further customized and or adapted for the individual.

The collected data and all information stored in device 102 may also be transmitted via network 106 (e.g., the Internet, a local area network (LAN), a wide area network (WAN), etc.) or via a direct connection between device 102 and server 106. All data/information may be communicated via a wireless connection, a wired connection, or some combination of both.

Analysis server 108 may be a back-end server that is used for more in-depth or historical processing and analysis of the data. Further analysis of the data may also be conducted via therapist 110 and/or researcher 112 and may include patient medical information stored on analysis server 108 or an information system networked to server 108. The results of the more in-depth or historical processing and analysis may be forwarded to mobile therapeutic device 102 as a feedback in order to adapt and improve the overall effectiveness of the administered mobile interventions.

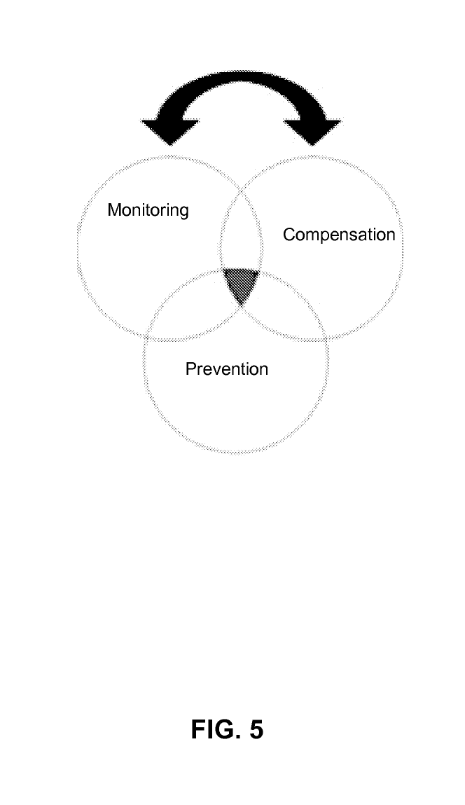

Embodiments of the invention may also determine when the individual handles feelings of anger or stress better than he or she did in the past. Here, positive reinforcement/encouragement is provided to the individual by mobile therapeutic device 102. In embodiments, the invention tracks the patient's trends of physiological patterns that indicate improved or worsened handling of anger or stress based on changes in physiological stress as detected by the system. This allows for the resetting of the patient's baseline. For example, after several weeks of treatment, the patient's heart rate variability may be higher due to increased coping skills, or lower due to increased health problems. This resetting of the patient's baseline is based on principles of adaptive learning. Principles of embedded assessment allow for the recalibration of feedback based on adaptive learning. FIG. 5 illustrates the dynamic relationship between continuous monitoring and feedback in the embedded assessment approach. Each of the components or elements of system 100 will be discussed next in more detail

In various embodiments, system 100 may be implemented as a wireless system, a wired system, or a combination of both. When implemented as a wireless system, system 100 may include components and interfaces suitable for communicating over a wireless shared media, such as one or more antennas, transmitters, receivers, transceivers, amplifiers, filters, control logic, and so forth. An example of wireless shared data may include portions of a wireless spectrum, such as the RF spectrum and so forth. When implemented as a wired system, system 100 may include components and interfaces suitable for communicating over wired communications media, such as input/output (I/O) adapters, physical connectors to connect the I/O adapter with a corresponding wired communications medium, a network interface card (NIC), disc controller, video controller, audio controller, and so forth. Examples of wired communications media may include a wire, cable, metal leads, printed circuit board (PCB), backplane, switch fabric, semiconductor material, twisted-pair wire, co-axial cable, fiber optics, and so forth.

As discussed above, real-time data is continuously collected for an individual via heart indicator 104. The collected data may be wirelessly transmitted to mobile therapeutic device 102 via, for example, Bluetooth technology, Zigbee technology or a proprietary system. The invention is not limited to these example wireless technologies. Alternatively, indicator 104 may transmit data to device 102 via a wired connection, or some combination of wireless and wired connection technologies.

Indicator 104 may also be adapted to store real-time data via integrated long term storage, such as flash memory for example, and then transmit the data to mobile therapeutic device 102 at a later time. The integrated long term storage helps to ensure that no collected data are lost if there is no connection currently available with device 102.

An additional benefit of the invention is that the form factors for the monitoring of data and the administration of the mobile interventions are non-stigmatizing. Thus, the invention may especially appeal to individuals who are concerned about health and wellness, but do not want to announce his or her concerns publicly.

In an embodiment of the invention, heart indicator 104 may include a small form factor of a wireless ECG to measure heart rate. In an embodiment of the invention, indicator 104 is an integrated physiological monitor worn by an individual as a wireless chest worn sensor. The sensor may communicate with mobile therapeutic device 102 via a Body Area Network (BAN)—a short-range wireless network to transmit monitored data.

As discussed above, mobile therapeutic device 102 receives real-time (or stored) data via heart indicator 104. Device 102 processes the data to determine whether the individual is currently experiencing (or about to experience) feelings of anger or stress. The data may also be processed by heart indicator 104 (e.g., ECG device) and/or by analysis server 108. If it is determined that the individual is currently experiencing (or about to experience) feelings of anger or stress, mobile therapeutic device 102 determines an appropriate mobile intervention. The administered mobile intervention via device 102 provides therapeutic feedback to the individual on a real-time basis either before, during or directly after feelings of anger or stress.

In one embodiment, mobile therapeutic device 102 may be any mobile device capable of performing the functionality of the invention described herein. Device 102 may be implemented as part of a wired communication system, a wireless communication system, or a combination of both. In one embodiment, for example, device 102 may be implemented as a mobile computing device having wireless capabilities. A mobile computing device may refer to any device having a processing system and a mobile power source or supply, such as one or more batteries, for example.

Examples of embodiments of a mobile computing device that may be adapted to include the functionality of the present invention include a laptop computer, ultra-laptop computer, portable computer, handheld computer, palmtop computer, personal digital assistant (PDA), cellular telephone, combination cellular telephone/PDA, smart phone, pager, one-way pager, two-way pager, messaging device, data communication device, and so forth.

Examples of such a mobile computing device also may include computers that are arranged to be worn by a person, such as a wrist computer, finger computer, ring computer, eyeglass computer, belt-clip computer, arm-band computer, shoe computers, clothing computers, and other wearable computers.

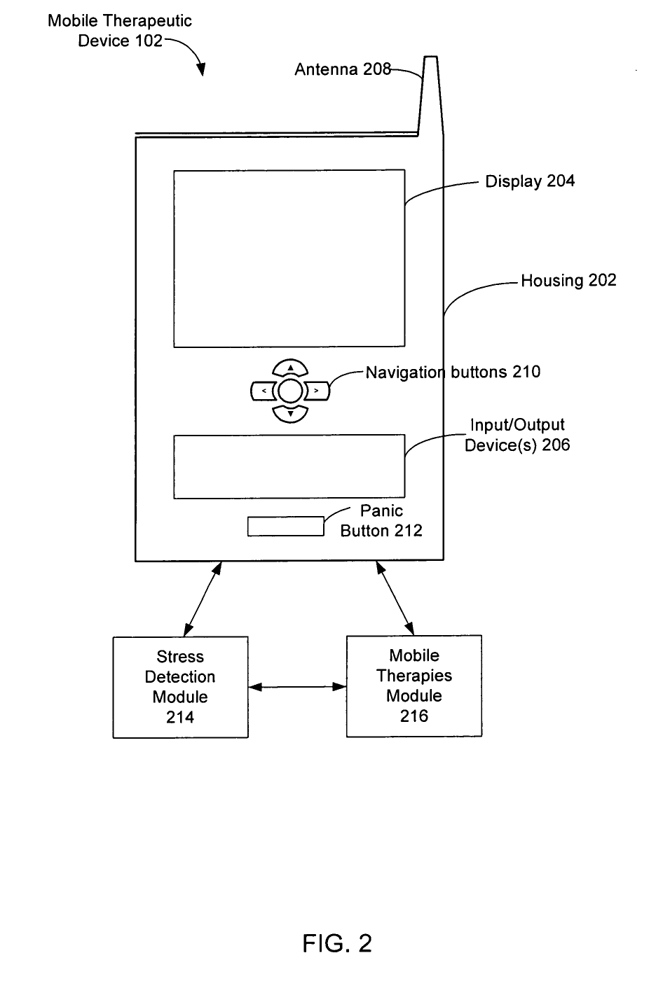

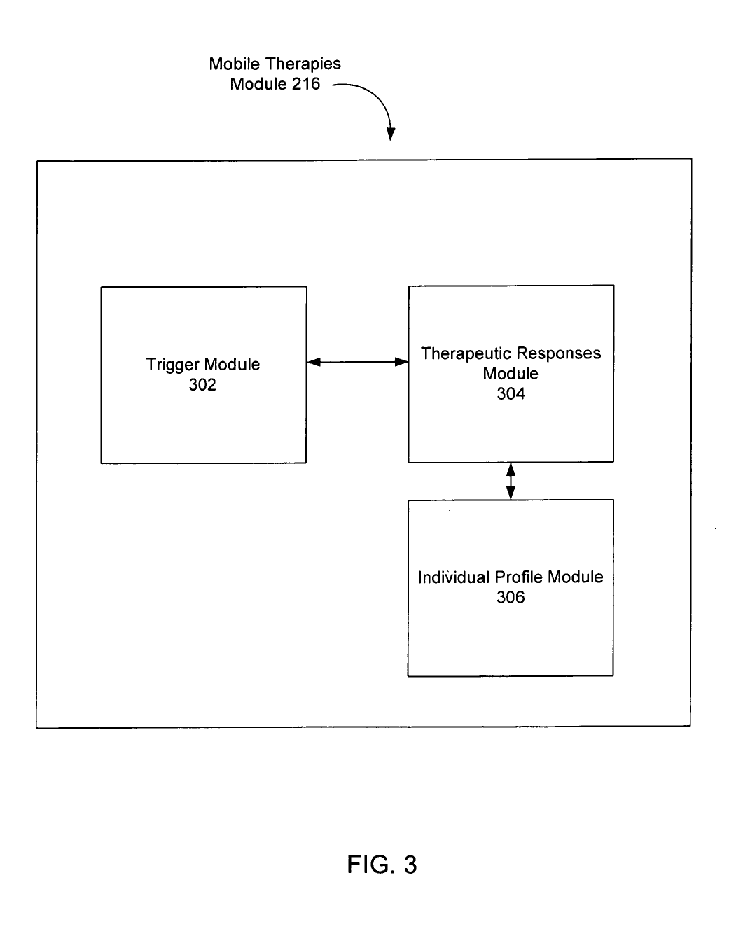

A more detailed description of an embodiment of mobile therapy device 102 is shown in FIGS. 2 and 3. Referring to FIG. 2, device 102 may include a housing 202, a display 204, one or more input/output devices 206, an antenna 208, navigation buttons 210, a panic button 212, an anger detection module 214 and a mobile therapies module 216.

Stress detection module 214 and/or mobile therapies module 216 may be integrated into device 102 or may be coupled to device 102 via a connection (e.g., wireless, wired or some combination of both). Note that although the functionality of modules 214 and 216 is described herein as being separated into two components, this is not meant to limit the invention. In fact, this functionality may be combined into one component or separated into three or more components. Additionally, one or both of anger detection module 214 and mobile therapies module 216 may be customized for an individual. Each of the components of FIG. 2 is described next in more detail.

Housing 202 may comprise any suitable housing, but typically involves a small form factor to enable mobile therapeutic device 102 to be easily transportable.

Display 204 may comprise any suitable display unit for displaying information appropriate for a mobile computing device. Display 204 is used by the invention to display mobile interventions to the individual, to assist with input into device 102, and so forth.

I/O device(s) 206 may comprise any suitable I/O device for entering information into and receiving information from mobile computing device 102. In embodiments of the invention, input is gathered implicitly from physiological monitoring and via touching iconic images on a screen to indicate ratings, for example. Input may also be gathered by gestures (e.g., turning mobile therapeutic device 102 upside down to indicate state of mind, etc.).

Examples for I/O device(s) 206 may include a suitable alphanumeric keyboard, a numeric keypad, a touch pad, input keys, buttons, switches, rocker switches, a microphone, a speaker, voice recognition device and software, and so forth. Information may be entered into device 102 by way of microphone. Such information may be digitized by a voice recognition device. The embodiments are not limited in this context.

Antenna 208 is used to facilitate wireless communication with mobile therapeutic device 102.

In one embodiment, navigation buttons 210 comprise an upward navigation button, a downward navigation button, a leftward navigation button, and a rightward navigation button. Navigation buttons 210 also may comprise a select button to execute a particular function on mobile therapeutic device 102.

Mobile interventions can be initiated by the user in several ways. For example, a menu allows quick access to breathing and relaxation exercises and a “panic button” (e.g., panic button 212) prompts a phone call that provides the user with a socially acceptable excuse to leave a negative situation.

As described above, stress detection module 214 processes the data sent from heart indicator 104 to determine potential feelings of anger or stress in the individual. If so, mobile therapies module 216 determines an appropriate intervention to enhance cognitive behavioral therapy for anger or stress reduction. The administered mobile intervention via device 102 provides therapeutic feedback to the individual on a real-time basis either right before, during or directly the feelings of anger or stress in the individual.

FIG. 3 illustrates one embodiment of mobile therapies module 216. Referring to FIG. 3, module 216 may comprise a trigger module 302, a therapeutic responses module 304 and an individual profile module 306. Note that although the functionality of modules 302, 304 and 306 is described herein as being separated into three components, this is not meant to limit the invention. In fact, this functionality may be combined into one or two components, or separated into four or more components. Trigger module 302, therapeutic responses module 304 and individual profile module 306 all may be customized to an individual, as will be described in more detail below.

Trigger module 302 processes the output of stress detection module 214 (FIG. 2) to determine one or more possible mobile interventions to administer on mobile therapeutic device 102. For example, assume that the output of stress detection module 214 indicates that the individual is experiencing feelings of anger or stress. Further assume that device 102 is provided information that the individual is currently driving his or her car. Here, so as to not impair the driving ability of the individual, trigger module 302 may determine that an audio intervention should be administered over a visual intervention.

As discussed above, a mobile intervention is supportive feedback administered via a mobile device on a real-time basis either right before, during or directly after the detection of anger or stress. One or more mobile interventions may be defined and stored in therapeutic responses module 304.

Mobile interventions may include, but are not limited to, interventions such as references to cognitive behavioral therapy techniques adapted for mobile therapeutic device 102, biofeedback, progressive muscle relaxation exercises, timely presentation of personal media (e.g., music and images collected from users), offers of an exit strategy (e.g., a phone call that helps the user escape from a stressful situation), references to a range of psychotherapeutic techniques and graphical representations of trends (e.g., depictions of health metrics over time), visual (e.g., picture), delay tactics (e.g., hourglass animation), breathing exercises, audio (e.g., music or humorous quotes) and cognitive reframing. These example mobile interventions are provided for illustration purposes only and are not meant to limit the invention.

An exit strategy may include a phone ring or a vibration administered via device 102 that allows an individual to excuse him or herself from a stressful situation. Visual or picture interventions display a visual cue to the individual that helps him or her deal with the challenges of stress. Audio interventions involve a clip of music or humorous quotes being played for the individual via device 102. The clips of music or humorous quotes are meant to relax the individual.

A range of mobile interventions help the user delay gratification of problematic behaviors. One example is an hourglass animation that could help someone to wait a certain length of time before submitting to a cigarette craving, eating a donut or even the temptation to get involved in a destructive confrontation. Often by waiting a short while, people can realize that they don't really need the cigarette, donut, or to engage in other problematic behavior.

Biofeedback displays on mobile therapeutic device 102 (e.g., a phone) raise self-awareness and mindfulness about health by showing an individual immediate data on physiological functioning (from the indicator(s)).

Cognitive reframing is a process in which individuals critically evaluate automatic thoughts and interpretations that are maladaptive. Cognitive reframing involves practices that the individual is learning in a clinical or self-help setting. Here, the mobile application (“mind scan”) reminds the user to question their interpretations.

Progressive muscle relaxation is a therapeutic technique in which individuals tense and relax different areas of the body—one at a time. For example, a phone display (“body scan”) walks the individual through the major muscle groups and assists in the tensing and relaxation of relevant muscles.

A presentation of visual trends and other analysis allows individual to view patterns of physical and emotional health indicators over time and their correlation with contextual factors. Visual stimuli are displayed on mobile therapeutic device 102. Clinicians can view these during and before treatment to modulate treatment and monitor patients. Queries on the database allow for different visual trends to be presented to the individual or clinician.

Referring back to FIG. 3, information stored in therapeutic responses module 304 for each intervention may include variations of the intervention itself, like escalations based on whether or not the individual responds physiologically to a given therapy, analogous to dosing or titrating of medication where the amount of medicine is tailored. For example, display picture_1 for 1 minute if individual is experiencing a decrease in heart rate variability below a first threshold that indicates the beginnings of a stressful period; display picture_2 for 2 minutes if individual is experiencing a decrease in heart rate variability below a second threshold that indicates moderate stress, and so on. Escalations in modality may also occur to encourage interaction with the system. For example, if the patient doesn't respond to a glowing of the phone display, the phone may vibrate and eventually chime. The broad range of variations to the mobile interventions are all assumed to be within the scope of the present invention.

In an embodiment of the invention, these variations of the mobile interventions may be set as determined by the medical profession regarding what is effective for certain health conditions and/or stressful situations.

In other embodiments, the variations of the mobile interventions may be adapted or customized for an individual according to what the present invention determines has been effective for the individual in the past to recover from feelings of anger or stress. In other embodiments, the invention might determine that what was most effective to reduce the feelings of anger or stress in an individual in the past is not working as well to date. Thus, the invention adapts and uses a different type of mobile intervention to better manage anger or stress in the individual.

As discussed above, embodiments of the present invention provide for adaptive learning and embedded assessment. In embodiments, the invention tracks the individual's trends in physiological patterns that indicate improved or worsened handling of health states based on changes in physiological stress as detected by the system. This allows resetting the baseline (after several weeks of treatment, an individual's heart rate variability may be higher due to increased coping skills, or lower due to increased health problems). This resetting of the patient's baseline is based on principles of adaptive learning. Principles of embedded assessment allow recalibration of feedback based on adaptive learning.

In other embodiments, the system personalizes interventions by incorporating personal information that is used by the invention and stored in individual profile module 306. For example, the individual may provide his or her own pictures, music clips, humorous quotes, mantras, timeframe for daily commutes, working hours and sleeping patterns, social support network, and so forth. Then, for example, when it is determined that audio intervention should be administered for an individual, the audio presented may be a music clip provided by the individual and stored in module 306 (versus one of the default music clips in module 304).

Operations for the above embodiments may be further described with reference to the following figures and accompanying examples. Some of the figures may include a logic flow. Although such figures presented herein may include a particular logic flow, it can be appreciated that the logic flow merely provides an example of how the general functionality as described herein can be implemented. Further, the given logic flow does not necessarily have to be executed in the order presented unless otherwise indicated. In addition, the given logic flow may be implemented by a hardware element, a software element executed by a processor, or any combination thereof.

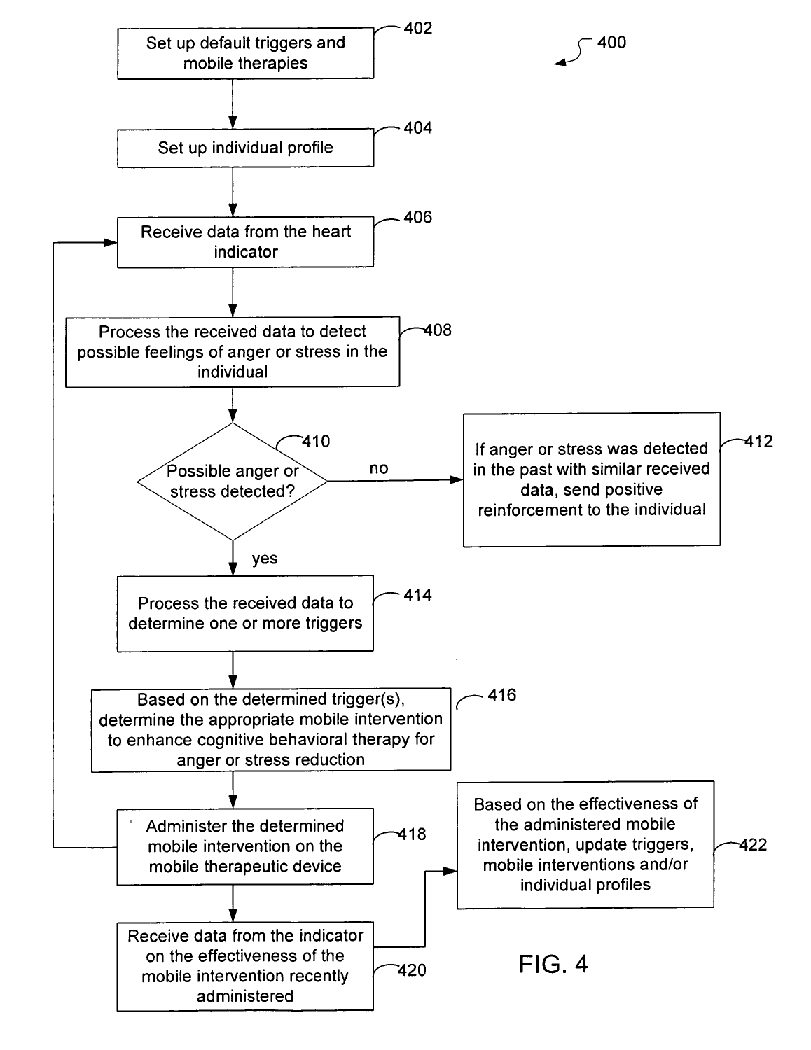

FIG. 4 illustrates one embodiment of a logic flow 400. The logic flow 400 may be representative of the operations executed by one or more embodiments described herein, for example, the operations executed by system 100.

Referring to FIG. 4, at block 402, initial default triggers and mobile interventions are set up in mobile therapeutic device 102. For example, default music clips may be defined for music interventions, default pictures may be defined for visual interventions, and so forth.

At block 404, an individual's profile is set up. This may include, but is not limited to, information provided by the individual. Such information may include, but is not necessarily limited to, pictures, music clips, humorous quotes, mantras, timeframe for daily commutes, working hours and sleeping patterns, social support network, and so forth.

At block 406, data is received by mobile therapeutic device 102. In an embodiment, the data received represents data collected about the individual via heart indicator 104, as described above.

At block 408, the received data is processed by mobile therapeutic device 102 to detect possible feelings of anger or stress in the individual. The data may also be processed by heart indicator 104 (e.g., ECG device) and/or by analysis server 108.

At block 410, if possible feelings of anger or stress are not detected, control passes to block 412. The invention is adapted to determine when the individual handles feelings of anger or stress better in the present than he or she did in the past. In block 412, it is determined whether feelings of anger or stress were determined in the past with similar received data for the individual. If so, then positive reinforcement/encouragement is provided to the individual by mobile therapeutic device 102 for handling feelings of anger or stress better now than in the past.

At block 410, if possible anger or stress is detected, control passes to block 414. In block 414, the received data is processed by mobile therapeutic device 102 to determine one or more triggers. As discussed above, triggers indicate one or more possible mobile interventions to administer on mobile therapeutic device 102.

At block 416, based on the determined triggers, mobile therapeutic device 102 determines the appropriate mobile intervention to administer to enhance cognitive behavioral therapy for anger or stress reduction in the individual. At block 418, the determined mobile intervention is administered on mobile therapeutic device 102.

Control then goes back to block 406, where the individual is continuously monitored via heart indicator 104.

At block 420, since the individual is continuously monitored, mobile therapeutic device 102 may analyze the possible feelings of anger or stress in the individual immediately after the administered mobile therapy to determine its effectiveness.

At block 422, data stored in mobile therapeutic device 102 and data stored in analysis server 112 may be updated to reflect the effectiveness of the administered mobile intervention.

Various embodiments may be implemented using hardware elements, software elements, or a combination of both. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Some embodiments may be implemented, for example, using a machine-readable or computer-readable medium or article which may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

Numerous specific details have been set forth herein to provide a thorough understanding of the embodiments. It will be understood by those skilled in the art, however, that the embodiments may be practiced without these specific details. In other instances, well-known operations, components and circuits have not been described in detail so as not to obscure the embodiments. It can be appreciated that the specific structural and functional details disclosed herein may be representative and do not necessarily limit the scope of the embodiments.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. An apparatus comprising:
a processor, a mobile power supply, at least one input device, and at least one output device; and
wherein the apparatus is capable of receiving data relating to a heart of an individual from the at least one input device, wherein the apparatus is capable of providing a mobile intervention via the at least one output device, and wherein the apparatus is capable of measuring the effectiveness of the mobile intervention in reducing anger or stress in the individual.

2. The apparatus of claim 1, wherein the apparatus is additionally capable of selecting the mobile intervention based on a previous effectiveness measurement.

3. The apparatus of claim 1, wherein the apparatus is additionally capable of recalibrating the mobile intervention based on adaptive learning.

4. The apparatus of claim 1, wherein the input device is a heart indicator that comprises a small form factor.

5. The apparatus of claim 1, wherein the mobile intervention comprises at least one of references to cognitive behavioral therapy techniques, biofeedback, breathing exercises, references to a range of psychotherapeutic techniques, delay tactics, and cognitive reframing.

6. The apparatus of claim 1, wherein the mobile intervention is provided to the individual either during or immediately after feelings of anger or stress.

7. The apparatus of claim 1, wherein the input device is a self-monitored indicator that comprises at least one of experience time sampling of mood and physical well being and journaling, wherein journaling allows the individual to report on his or her health states and related behaviors through one or more of responses to questionnaires and menu selection.

8. The apparatus of claim 7, wherein the experience time sampling includes prompting the user for responses at frequent time intervals of every thirty (30) minutes.

9. The apparatus of claim 7, wherein the experience time sampling may be triggered by one or more of changes in the heart and upon completion of the mobile intervention.

10. The apparatus of claim 1, wherein the received data is related to heart rate variability.

11. The apparatus of claim 1, wherein the received data is related to one of ECG amplitude, ST segment analysis and QT interval.

12. A method comprising:
receiving data relating to a heart of an individual from at least one input device;
providing a mobile intervention via at least one output device; and
measuring the effectiveness of the mobile intervention in reducing anger or stress in the individual.

13. The method of claim 12, further comprising selecting the mobile intervention based on a previous effectiveness measurement.

14. The method of claim 12, further comprising recalibrating the mobile intervention based on adaptive learning.

15. The method of claim 12, wherein the at least one input device is a heart indicator that comprises a small form factor.

16. The method of claim 12, wherein the mobile intervention comprises at least one of references to cognitive behavioral therapy techniques, biofeedback, breathing exercises, references to a range of psychotherapeutic techniques, delay tactics, and cognitive reframing.

17. The method of claim 12 wherein the mobile intervention is provided to the individual either during or immediately after feelings of anger or stress.

18. The method of claim 12, wherein the input device is a self-monitored indicator that comprises at least one of experience time sampling of mood and physical well being and journaling, wherein journaling allows the individual to report on his or her health states and related behaviors through one or more of responses to questionnaires and menu selection.

19. The method of claim 18, wherein the experience time sampling includes prompting the user for responses at frequent time intervals of every thirty (30) minutes.

20. The method of claim 18, wherein the experience time sampling may be triggered by one or more of changes in the heart and upon completion of the mobile intervention.

21. The method of claim 12, wherein the received data is related to heart rate variability.

22. The method of claim 12, wherein the received data is related to one of ECG amplitude, ST segment analysis and QT interval.

23. A machine-readable storage medium containing instructions which, when executed by a processing system, cause the processing system to perform instructions for:
receiving data relating to a heart of an individual from at least one input device;
providing a mobile intervention via at least one output device; and
measuring the effectiveness of the mobile intervention in reducing anger or stress in the individual.

24. The machine-readable storage medium of claim 23, further comprising selecting the mobile intervention based on a previous effectiveness measurement.

25. The machine-readable storage medium of claim 23, further comprising recalibrating the mobile intervention based on adaptive learning.

26. The machine-readable storage medium of claim 23, wherein the at least one input device is a heart indicator that comprises a small form factor.

27. The machine-readable storage medium of 23, wherein the mobile intervention comprises at least one of references to cognitive behavioral therapy techniques, biofeedback, breathing exercises, references to a range of psychotherapeutic techniques, delay tactics, and cognitive reframing.

28. The machine-readable storage medium of 23, wherein the mobile intervention is provided to the individual either during or immediately after feelings of anger or stress.

29. The machine-readable storage medium of 23, wherein the input device is a self-monitored indicator that comprises at least one of experience time sampling of mood and physical well being and journaling, wherein journaling allows the individual to report on his or her health states and related behaviors through one or more of responses to questionnaires and menu selection.

30. The machine-readable storage medium of 29, wherein the experience time sampling includes prompting the user for responses at frequent time intervals of every thirty (30) minutes.

31. The machine-readable storage medium of 29, wherein the experience time sampling may be triggered by one or more of changes in the heart and upon completion of the mobile intervention.

32. The machine-readable storage medium of 23, wherein the received data is related to heart rate variability.

33. The machine-readable storage medium of 23, wherein the received data is related to one of ECG amplitude, ST segment analysis and QT interval.
